(19) United States
(12) Patent Application Publication
(10) Pub. No.: US 2014/0358172 A1
(43) Pub. Date: Dec. 4, 2014

(54) EASY-TO-GRIP ANTI-SPLASH NEEDLE MOUNTING STRUCTURE

(71) Applicant: MEI-CHI-NA HSINYEN CO., LTD., Taipei City (TW)

(72) Inventor: Su-Lin LIN, Taipei City (TW)

(73) Assignee: MEI-CHI-NA HSINYEN CO., LTD., Taipei City (TW)

(21) Appl. No.: 13/905,219

(22) Filed: May 30, 2013

(51) Int. Cl. A61B 17/34; A61M 37/00

(52) U.S. Cl. CPC A61B 17/3403; A61M 37/0076; A61B 2017/0042
USPC 606/185

(57) ABSTRACT

The present invention is to provide an anti-splash needle mounting structure, which includes a grip portion having a receiving space therein for receiving a tattoo needle driving mechanism and an opening at a bottom end thereof in communication with the receiving space so that a tattoo needle of the tattoo needle driving mechanism can be exposed outside the opening, and a tattoo needle guide tube having two ends provided with two through holes respectively and an outer periphery of the top end thereof provided with a second connecting unit configured for connection with a first connecting unit provided in an inner periphery of the opening. The two through holes are in communication with each other, and the inner wall of the tattoo needle guide tube is axially formed with a plurality of grooves each having two ends extending to the two through holes respectively.

### FIELD OF THE INVENTION

The present invention relates a needle mounting structure, more particularly to an anti-splash needle mounting structure installed on an eyebrow tattoo machine, so as not only to allow an eyebrow tattooist to firmly grip a grip portion of the eyebrow tattoo machine, but also to allow the ink on the tip of a tattoo needle to be temporarily stored in a plurality of grooves inside the needle mounting structure due to the capillary action of the ink while the tattoo needle moves back and forth in the needle mounting structure, thereby enabling the ink to flow along the grooves and coat onto the surface of the tattoo needle evenly, and effectively preventing excessive ink from gushing or splashing out of the needle mounting structure and tainting or injuring the eyes or the surrounding skin of the person being tattooed.

### BACKGROUND OF THE INVENTION

To women in the modern society, eyebrow tattooing means more than saving the time required for daily makeup application. Eyebrows can define a person's countenance—be it a happy, angry, or sad one—or even show different characters. Given today's cosmetic surgery techniques, eyebrows are the only facial feature that can be reshaped without causing much physical pain. If eyes are a window to the soul, then eyebrows, which allow modification to the greatest extent as compared with the other facial features, can be said to be the light above that window, for a pair of nicely shaped eyebrows can definitely accentuate the beauty of the eyes. This is the main reason why eyebrow tattooing has become increasingly popular.

Traditionally, eyebrow tattooing was carried out solely by hand, and if the pigment was not implanted to a uniform depth, the resultant eyebrow tattoos might look asymmetric or have an uneven shade. To overcome the various drawbacks of the traditional manual eyebrow tattooing method, mechanical eyebrow tattoo machines were developed to make more natural-looking eyebrow tattoos.

Generally speaking, a conventional eyebrow tattoo machine consists of a transmission mechanism and a needle mounting mechanism, wherein the transmission mechanism is configured for driving the tattoo needle in the needle mounting mechanism into reciprocal up-and-down movement along the central axis of the eyebrow tattoo machine. During the tattooing process, an eyebrow tattooist must hold the grip portion of the needle mount tightly with his or her fingers and thumb. However, as the grip portion of the needle mount is tubular, the eyebrow tattooist may find it difficult to hold the grip portion firmly after having held it for a long time, for any sweat or ink on the fingers or thumb will make the grip portion very slippery. Should the eyebrow tattooist have problem gripping the needle mount securely, errors may occur in tattooing. Moreover, although eyebrow tattoos are made on a person's face by forming predetermined patterns of the person's desired shapes with the ink on the tattoo needle, the ink tends to be brought into the needle mount as the tattoo needle moves reciprocally up and down during the tattooing process, and the ink clinging on the inner wall of the needle mount will gather together due to “cohesion” of the ink and then gush or splash out of the needle mount as a result of the vertical reciprocating movement of the tattoo needle, thus tainting or even injuring the eyes or the surrounding skin of the person being tattooed. “Cohesion” is the force whereby like molecules, e.g., water molecules, are attracted to one another. The accumulation of ink in the needle mount can be viewed as the gathering of water droplets on a piece of glass. As the water droplets gather together to form a large water drop, so does the ink in the needle mount. The large ink drop thus formed will eventually be driven out of the needle mount in a gushing or splashing manner by the vertically reciprocating tattoo needle.

Therefore, the issue to be addressed by the present invention is to design a needle mounting structure capable of solving the aforementioned problems. It is desirable that not only can an eyebrow tattooist firmly hold the grip portion of an eyebrow tattoo machine during tattooing, but also there will be no excessive ink gushing or splashing out of a tattoo needle guide tube when a tattoo needle coated with ink moves back and forth in the needle mounting structure, thereby preventing the ink from tainting or injuring the eyes or the surrounding skin of the person being tattooed.

### BRIEF SUMMARY OF THE INVENTION

In view of the drawbacks of the needle mounting structure of the aforesaid eyebrow tattoo machine (e.g., the difficulty with which an eyebrow tattooist can firmly hold the grip portion of the needle mounting structure when the grip portion has been made slippery by the sweat on the tattooist's hand after a prolonged grip or by any ink on the tattooist's hand; potential mistakes in tattooing due to such difficult gripping; the gushing or splashing of ink from inside a tattoo needle guide tube of the needle mounting structure as a result of an ink-coated tattoo needle moving reciprocally in the tattoo needle guide tube; and the chances of such gushing or splashing ink tainting or injuring the eyes or the skin surrounding the eyes of the person being tattooed) the inventor of the present invention conducted extensive research and experiment and finally succeeded in developing a novel needle mounting structure as disclosed herein for effectively solving the problems cited above.

It is an object of the present invention to provide an easy-to-grip anti-splash needle mounting structure. The needle mounting structure is installed on an eyebrow tattoo machine and includes a grip portion and a tattoo needle guide tube. The grip portion is a tube provided therein with a receiving space for receiving a tattoo needle driving mechanism. The bottom end of the grip portion is provided with an opening in communication with the receiving space so that a tattoo needle of the tattoo needle driving mechanism can be exposed outside the opening. The inner periphery of the opening is provided with a first connecting unit (e.g., internal threads, a rib, or a recess) extending toward (or away from, in the case of a recess) the receiving space. The outer periphery of the grip portion has a concave part corresponding in position to the first connecting unit. The concave part is shaped to be closely gripped by an eyebrow tattooist's fingers and thumb, thus enabling the eyebrow tattooist to hold the grip portion firmly. The top end of the tattoo needle guide tube is provided with a first through hole, and the bottom end of the tattoo needle guide tube is provided with a second through hole in communication with the first through hole. The outer periphery of the top end of the tattoo needle guide tube is provided with a second connecting unit (e.g., external threads, a recess, or a rib) configured for connection with the first connecting unit of the grip portion. Once the first and the second connecting units are connected to each other, the top end of the tattoo needle guide tube is connected to the bottom end of the grip portion, so as for the tattoo needle to extend through both the through holes, and the tip of the tattoo needle to be exposed outside the second through hole. In addition, the inner wall of the tattoo needle guide tube is axially formed with a plurality of grooves. Each groove has two ends extending to the first and the second through holes of the tattoo needle guide tube respectively.

Thus, not only can an eyebrow tattooist have a firm grip of the grip portion, but also the ink on the tip of the tattoo needle can be temporarily stored in the grooves due to the capillary action of the ink therein while the tattoo needle moves back and forth in the needle mounting structure. The ink will flow along the grooves and coat the surface of the tattoo needle evenly, thereby enabling a uniform depth of ink insertion and hence a uniform size and shade of the resultant eyebrow tattoos. Furthermore, excessive ink is effectively prevented from gushing or splashing out of the tattoo needle guide tube and is thus prevented from tainting or injuring the eyes or the surrounding skin of the person being tattooed.

The structure as well as a preferred mode of use, further objects, and advantages of the present invention will be best understood by referring to the following detailed description of some illustrative embodiments in conjunction with the accompanying drawings, in which:

### BRIEF DESCRIPTION OF THE SEVERAL VIEWS OF THE DRAWINGS

FIG. 1 is a sectional view of a preferred embodiment of the present invention;

FIG. 2 is an exploded sectional perspective view of the preferred embodiment of the present invention; and

FIG. 3 is an exploded sectional view of another preferred embodiment of the present invention.

### DETAILED DESCRIPTION OF THE INVENTION

“Capillary action” refers to the movement of a liquid in a thin tube or tiny gap opening at two ends and occurs in the following manner. When a liquid in a thin tube shows lower cohesion than adhesion with the tube wall, the liquid will attach to the tube wall and move upward against gravity. One common example of “capillary action” is the upward transportation of water from a plant's roots through the vascular bundles in the stem. “Capillary action” also results in the concavity or convexity of the free surface of a liquid in a thin vertical tube and the liquid absorptivity of a porous substance.

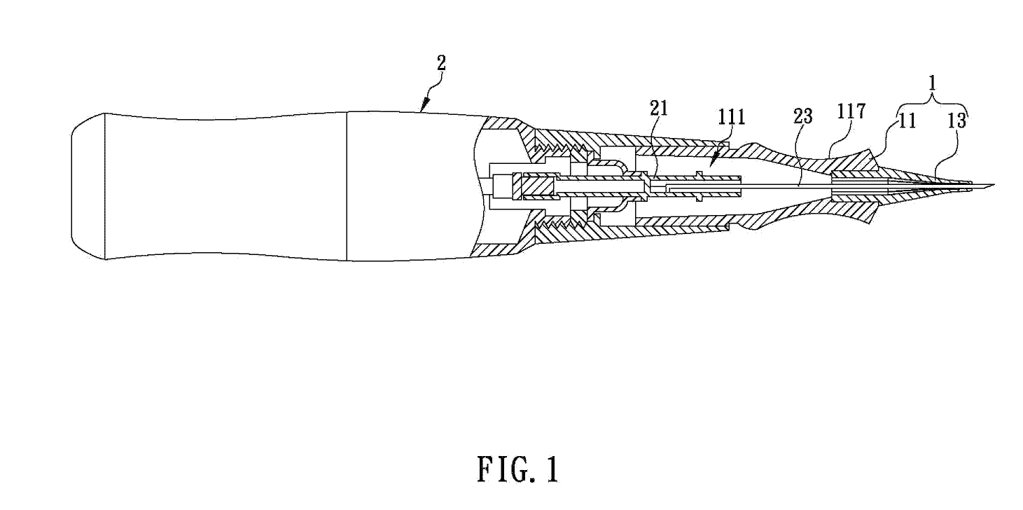

The needle mounting structure of the present invention is designed on the principle of “capillary action” stated above. Referring to FIG. 1 for the first preferred embodiment of the present invention, the needle mounting structure 1 is installed on an eyebrow tattoo machine 2 and includes a grip portion 11 and a tattoo needle guide tube 13. The grip portion 11 is a tube provided with a receiving space 111 therein for receiving a tattoo needle driving mechanism 21. The bottom end of the grip portion 11 is provided with an opening 113 in communication with the receiving space 111, thus allowing a tattoo needle 23 of the tattoo needle driving mechanism 21 to be exposed outside the opening 113. The inner periphery of the opening 113 is provided with a first connecting unit 115 (e.g., internal threads) extending toward the receiving space 111. The outer periphery of the grip portion 11 has a concave part 117 corresponding in position to the first connecting unit 115. The concave part 117 is so shaped that an eyebrow tattooist can grip it tightly with his or her fingers and thumb and thereby hold the grip portion 11 securely. Hence, even if the eyebrow tattooist's hand has sweat after gripping the grip portion 11 for a long time, or even if the eyebrow tattooist's hand is wetted with ink, the eyebrow tattooist can still have a firm grip on the grip portion 11 without feeling slippery and perform the tattoo operation accurately.

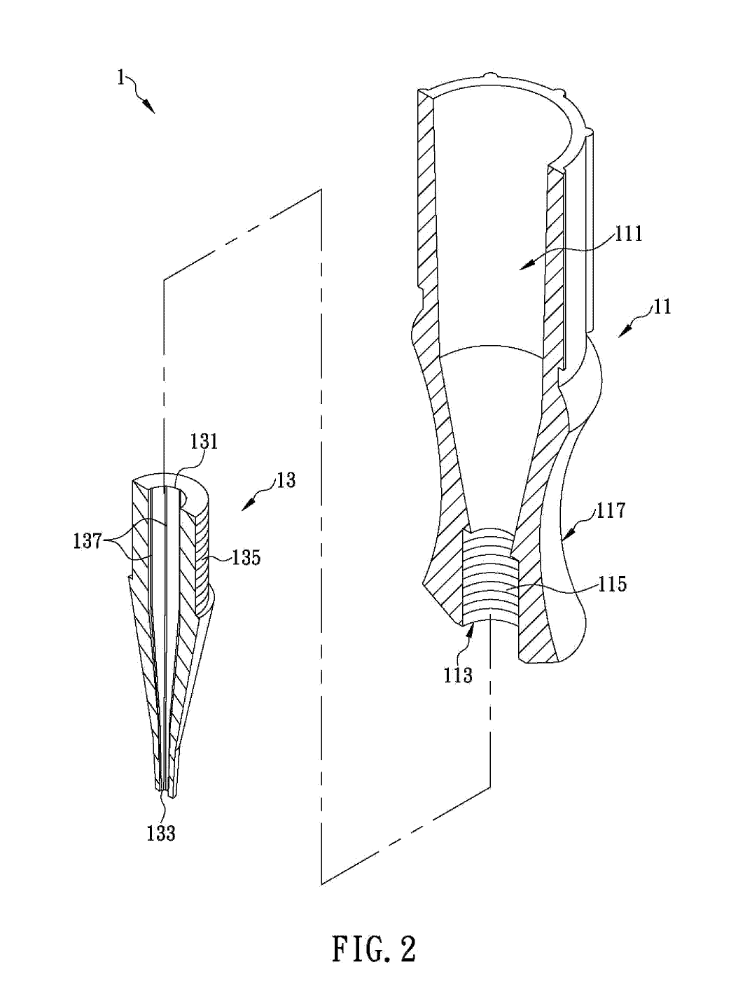

As shown in FIG. 1 and FIG. 2, the tattoo needle guide tube 13 is provided with a first through hole 131 and a second through hole 133 at its top end and bottom end respectively, wherein the first and the second through holes 131 and 133 are in communication with each other. The outer periphery of the top end of the tattoo needle guide tube 13 is provided with a second connecting unit 135 (e.g., external threads). The second connecting unit 135 is connectable with the first connecting unit 115 of the grip portion 11 so that the top end of the tattoo needle guide tube 13 can connect with the bottom end of the grip portion 11. Once such connection is made, the tattoo needle 23 can extend through both the first through hole 131 and the second through hole 133, so as for the tip of the tattoo needle 23 to be exposed outside the second through hole 133, allowing an eyebrow tattooist to coat the tip of the tattoo needle 23 with ink. The inner wall of the tattoo needle guide tube 13 is formed with a plurality of grooves 137 which extend in the axial direction of the tattoo needle guide tube 13. The two ends of each groove 137 extend to the first through hole 131 and the second through hole 133 of the tattoo needle guide tube 13 respectively.

Referring back to FIG. 1 and FIG. 2, when the tattoo needle 23 has its tip coated with ink and is moved back and forth in the needle mounting structure 1, the tip of the tattoo needle 23 and the ink on the tip are brought into the tattoo needle guide tube 13 repeatedly, and the ink will, due to its cohesion, gather on the inner wall of the tattoo needle guide tube 13. As the tattoo needle 23 continues moving reciprocally, capillary action of the ink takes place in the grooves 137, causing the ink to not only stay temporarily in, but also flows along, the grooves 137. Consequently, the ink is coated evenly on the surface of the tattoo needle 23 and can therefore be inserted evenly into the skin of the person being tattooed, making eyebrow tattoos that are uniform in shape and shade. Moreover, as excessive ink is effectively kept from gushing or splashing out of the tattoo needle guide tube 13, the eyes of the person being tattooed or the skin surrounding the eyes will not be tainted or injured by such excessive ink.

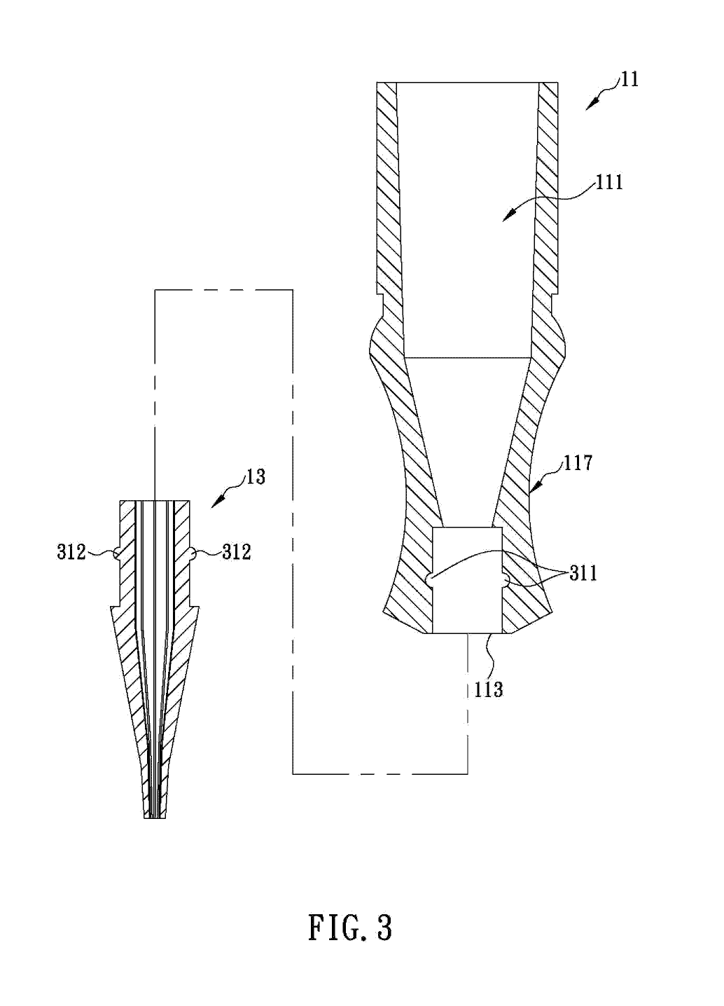

In the embodiment described above, the first connecting unit 115 is internal threads while the second connecting unit 135 is external threads. In other preferred embodiments of the present invention, however, the configurations of the connecting units are not limited to the foregoing and may be changed according to production requirements. Reference is now made to FIG. 3, in which all the reference numerals are the same as those used in the first preferred embodiment except for those of the connecting units. In the second preferred embodiment of the present invention as shown in FIG. 3, the grip portion 11 is provided therein with a receiving space 111 and has a bottom end provided with an opening 113. The opening 113 is in communication with the receiving space 111 and has an inner periphery provided with a first connecting unit 311 (e.g., a recess or a rib) extending away from or toward the receiving space 111. The outer periphery of the grip portion 11 has a concave part 117 corresponding in position to the first connecting unit 311. The concave part 117 is so shaped that an eyebrow tattooist can grip it tightly with his or her fingers and thumb and thereby hold the grip portion 11 firmly.

As shown in FIG. 3, the top end of the tattoo needle guide tube 13 has an outer periphery provided with a second connecting unit 312 (e.g., a rib or a recess). The second connecting unit 312 corresponds in configuration to the first connecting unit 311 of the grip portion 11, so as for the top end of the tattoo needle guide tube 13 to connect with the bottom end of the grip portion 11 as in the first preferred embodiment of the present invention.

While the invention herein disclosed has been described by means of specific embodiments, numerous modifications and variations could be made thereto by those skilled in the art without departing from the scope of the invention set forth in the claims.

What is claimed is:

1. An easy-to-grip anti-splash needle mounting structure, comprising:
a grip portion formed as a tube and provided therein with a receiving space for receiving a tattoo needle driving mechanism, the grip portion having a bottom end provided with an opening in communication with the receiving space, the opening having an inner periphery provided with a first connecting unit, the first connecting unit extending toward or away from the receiving space, the grip portion having an outer periphery formed with a concave part corresponding in position to the first connecting unit; and
a tattoo needle guide tube having a top end provided with a first through hole and a bottom end provided with a second through hole in communication with the first through hole, the top end of the tattoo needle guide tube having an outer periphery provided with a second connecting unit, the second connecting unit being connectable with the first connecting unit so that the top end of the tattoo needle guide tube is connectable with the bottom end of the grip portion, thus allowing a tattoo needle of the tattoo needle driving mechanism to extend through both said through holes, and a tip of the tattoo needle to be exposed outside the second through hole, the tattoo needle guide tube having an inner wall provided with a plurality of grooves extending in an axial direction of the tattoo needle guide tube, each said groove having two ends respectively extending to the first and the second through holes of the tattoo needle guide tube.

2. The needle mounting structure of claim 1, wherein the first connecting unit is configured as internal threads, and the second connecting unit is configured as external threads.

3. The needle mounting structure of claim 1, wherein the first connecting unit is configured as a recess, and the second connecting unit is configured as a rib.

4. The needle mounting structure of claim 1, wherein the first connecting unit is configured as a rib, and the second connecting unit is configured as a recess.
